(19) United States
(12) Patent Application Publication
(10) Pub. No.: US 2010/0317112 A1
(43) Pub. Date: Dec. 16, 2010

(54) SCAFFOLDS INCREASED SPECIFIC GRAVITY FOR CELL CULTURE AND METHOD FOR MANUFACTURING THEREOF

(76) Inventors: Hyunjin Yang, Seoul (KR); Heeyoung Lee, Jeollabuk-do (KR); Jun Seok Lee, Busan (KR)

Correspondence Address: Edwards Angell Palmer & Dodge LLP, P.O. Box 55874, Boston, MA, 02205, US

(21) Appl. No.: 12/597,895

(22) PCT Filed: Apr. 27, 2007
(86) PCT No.: PCT/KR2007/002065

§ 371 Date: Aug. 31, 2010

(51) Int. Cl. C12N 5/02

(52) U.S. Cl. 435/396

(57) ABSTRACT

The present invention relates to microtype scaffolds for cell culture, which have their specific gravity increased and a method for manufacturing thereof, and more specifically, relates to microtype scaffolds for cell culture, which have their specific gravity increased, by adding a chemically stable inorganic compound having a high specific gravity in manufacturing biocompatible polymer microtype scaffolds for cell culture and a method for manufacturing thereof. In case where the inventive microtype scaffolds for cell culture is used, it is easy to separate cells cultured on microtype scaffolds, and cell damage can be minimized by reducing separation time, and it is easy to recover cells due to a definite boundary layer.

### TECHNICAL FIELD

The present invention relates to microtype scaffolds for cell culture, which have their specific gravity increased and a method for manufacturing thereof, and more specifically, relates to microtype scaffolds for cell culture, which have their specific gravity increased, by adding a chemically stable inorganic compound having a high specific gravity in manufacturing biocompatible polymer microtype scaffolds for cell culture and a method for manufacturing thereof.

### BACKGROUND ART

Most cells except cancer cells grow, adhering to a culture substrate, and studies on culture plates and culture materials for increasing cell proliferation are being conducted. Moreover, cell culture methods, to which biocompatible microtype scaffold particles are applied to increase the surface area for cell adherence, are generally used. However, cells must be separated from microtype scaffolds to be recovered, and when cells are separated by centrifugation which is the most appropriate separation method, there are problems in that the separation time is increased because the difference between specific gravity of microtype scaffolds and that of cells is not significant, which causes cell damage, and there is a high possibility of cell loss due to unclear boundary between microtype scaffolds and cell layers.

Various methods for cell culture have been introduced in the field of adult stem cells, but they have a problem of increasing cell culture efficiency. As an attempt to solve the problem, there is a three-dimensional culture method using micro-scaffolds which has an increased cell adhesion probability by maximization of cell adhesion area through micronization of microtype scaffolds and intermittent relative motion (Korean patent application No. 2006-79725).

In the past, culture methods using microtype scaffolds having a diameter of more than 100 μm, which are relatively larger than that of cells, were introduced but recently, since microtype scaffolds having a diameter of 10˜100 μm are being used, filters for cell separation must be micronized. However, since the smaller the size of a filter is, the higher the possibility of time consumption and cell loss is, it is more practical to use centrifugation compared to microfilter method. However, there are disadvantages in that centrifugation time is extended in the process of cell separation after final trypsin treatment because there is no significant difference between specific gravity of polymer microtype scaffolds used mainly in culture and that of cells, and cells are lost since it is difficult to detect a definite boundary layer.

Centrifugation is one of the processes which are the most frequently used in the biotechnology field dealing mainly with cells. However, there is a limitation on separation rate as there is a limitation on the centrifugal force to prevent cell destruction, and time (about 100 G (separation capability, the number in proportion to RCF (relative centrifugal force))/10 minutes). At this time, the greater the difference in specific gravities is, the easier it is to separate cells from microtype scaffolds even with relatively low RCF. Especially, when specific gravity solutions, such as Percoll, Ficoll, Hyperfaque etc., are used as means for detecting a definite boundary layer upon separation of nucleate cells, such as stem cells, B-lymphocytes, a boundary between a cell layer and its upper layer can be easily detected and thus they are frequently used in centrifugation. The use of said specific gravity solution in manufacturing microtype scaffolds for cell culture is to easily obtain a boundary layer, which lead to an increase in easiness of centrifugation.

Microtype scaffolds for cell culture have been limited to a minimum number of materials depending on cell characteristics and the purpose thereof for safe and efficient cell culture. The materials generally used for manufacturing microtype scaffolds are natural or synthetic polymer materials whose specific gravity is similar to that of cells. Clinically approved biodegradable or biocompatible materials used at present are a few polysaccharides, such as polylactic acid (PLA), poly L-lactic acid (PLLA), poly glycolic acid (PGA), poly lactic-co-glycolic acid (PLGA), polycapro-lactam (PCL) etc. Their specific gravity is determined in the range of 1.1˜1.3, and specific gravity of cells or solid materials derived from human is also more than 1.2, and thus there is a problem in that they require centrifugation process because specific gravity of polymer is similar to that of cells or solid materials derived from human.

If there is no significant difference in specific gravity, a solution of a desired specific gravity are usually used, like distinction of jewelries or seeds etc., but there are limitations in using without damaging cells. Since most widely used specific gravity solutions generally have specific gravity of less than 1.2, it is difficult to find specific gravity solutions which are used to identify the boundary between nucleate cells having specific gravity of more than 1.2 and microtype scaffolds. Especially, in case where they are injected into the human body as a cell treatment agent, it is not easy to use in clinical practice since it is necessary to test so many times and it is more likely to be the target of regulation.

Therefore, since the problems occurring in centrifugation will be solved if specific gravity of a microtype scaffold itself is increased, it will not be difficult to reduce the time required for centrifugation and to detect the boundary layer if a polymer material is used as a basic material for a microtype scaffold and specific gravity of the microtype scaffold is increased to more than 1.3.

Accordingly, the present inventors have made extensive efforts to develop microtype scaffolds for cell culture which are easy to collect separated cells, and as a result, confirmed that when specific gravity of a microtype scaffold is increased compared to existing microtype scaffolds for cell culture by adding an inorganic compound ingredient, which have a high specific gravity, to microtype scaffolds for cell culture, cells can be separated only by centrifugation and cell damage can be minimized, thereby completing the present invention.

### SUMMARY OF THE INVENTION

The main object of the present invention is to provide a method for manufacturing microtype scaffolds for cell culture, which have increased specific gravity compared to existing microtype scaffolds for cell culture.

Another object of the present invention is to provide a microtype scaffold for cell culture comprising a biocompatible polymer and an inorganic compound and a method for culturing cells using said microtype scaffold for cell culture.

To achieve the above objects, the present invention provides a method for manufacturing microtype scaffolds for cell culture, the method comprising the steps of: (a) mixing an inorganic compound with a biocompatible polymer; and (b) mixing a biocompatible polymer with the mixture obtained in the step (a), and then washing and drying, thus manufacturing microtype scaffolds.

The present invention also provides a method for manufacturing microtype scaffolds for cell culture, the method comprising the steps of: (a) mixing two or more biocompatible polymers; and (b) mixing an inorganic compound with the mixture obtained in the step (a), and then washing and drying, thus manufacturing microtype scaffolds.

The present invention also provides a method for manufacturing microtype scaffolds for cell culture, the method comprising the steps of: (a) mixing two or more biocompatible polymers; and (b) washing and drying the mixture obtained in the step (a), and then coating it with an inorganic compound, thus manufacturing microtype scaffolds.

The present invention also provides a microtype scaffold for cell culture prepared by the method, which contains a biocompatible polymer and an inorganic compound for increasing specific gravity and has a diameter of 10250 μm, and a method for culturing cells, which comprises using said microtype scaffold for cell culture.

Other features and embodiments of the present invention will be more fully apparent from the following detailed description and appended claims.

### BRIEF DESCRIPTION OF DRAWINGS

FIG. 1 is a photograph of scanning electron microscopy showing microtype scaffolds for cell culture, having increased specific gravity, which is manufactured using indium oxide.

FIG. 2 is a photograph of scanning electron microscopy showing microtype scaffolds for cell culture, having increased specific gravity, which is manufactured using titanium oxide.

### DETAILED DESCRIPTION OF THE INVENTION, AND PREFERRED EMBODIMENTS

In one aspect, the present invention relates to a method for manufacturing microtype scaffolds for cell culture, which have increased specific gravity.

In one embodiment of the method for manufacturing a microtype scaffold for cell culture according to the present invention, the method comprises the steps of: (a) mixing an inorganic compound with a biocompatible polymer; and (b) mixing a biocompatible polymer with the mixture obtained in the step (a), and then washing and drying, thus manufacturing a microtype scaffold.

In another embodiment of the method for manufacturing a microtype scaffold for cell culture according to the present invention, the method comprises the steps of: (a) mixing two or more biocompatible polymers; and (b) mixing an inorganic compound with the mixture obtained in the step (a), and then washing and drying, thus manufacturing a microtype scaffold.

In still another embodiment of the a method for manufacturing a microtype scaffold for cell culture according to the present invention, the method comprises the steps of: (a) mixing two or more biocompatible polymers; and (b) washing and drying the mixture obtained in the step (a), and then coating it with an inorganic compound, thus manufacturing a microtype scaffold.

The methods for manufacturing microtype scaffolds for cell culture according to embodiments of the present invention additionally comprise a step (c) of: collecting the microtype scaffolds by using a specific gravity solution.

In the present invention, the biocompatible polymer is preferably selected from the group consisting of poly lactic acid (PLA), poly L-lactic acid (PLLA), poly glycolic acid (PGA), poly lactic-co-glycolic acid (PLGA), polyvinylalcohol (PVA), collagen, alginate, chitosan, fluorine resin(Teflon), agar gel and polyacrylamide, and the inorganic compound is preferably selected from the group consisting of ceramic or metal.

In the present invention, the ceramic is preferably selected from the group consisting of hydroxyapatite (Ca₁₀(POᵣ)₆(OH)₃), titanium dioxide (TiO₂), barium titanate (BiTiO₃), zircon (ZrSiO₄), zirconia dioxide (ZrO₂), iron oxide, zinc oxide (ZnO), silicon dioxide (SiO₂), indium oxide (In₂O₃) and tin oxide (SnO₂), and the metal is preferably selected from the group consisting of calcium, phosphorus, titanium, zirconium (Zr), iron (Fe), zinc, silicon, indium (In) and tin (Ti).

In the present invention, the inorganic compound is preferably a compound responding to light, and the compound responding to light is preferably selected from the group consisting of titanium dioxide (TiO₂), zinc oxide (ZnO) and tin oxide (SnO₂). In case of manufacturing microtype scaffolds for cell culture using the inorganic compound responding to light, cell adherence can be regulated by varying radiation intensity. In other words, since titanium dioxide etc. responds to light, cell adherence can be regulated by controlling radiation intensity when cells are cultured by using microtype scaffolds for cell culture, which were manufactured using an inorganic compound, such as titanium dioxide etc. responding to light.

In the present invention, to manufacture microtype scaffolds for cell culture, which have increased specific gravity, indium oxide (In₂O₃) or titanium dioxide (TiO₂) was added to a biocompatible polymer poly L-lactide (PLLA) to stir, and then the resulting mixture was added with polyvinylalcohol (PVA) to stir, thus manufacturing microtype scaffolds, or polyvinylalcohol, poly L-lactide and indium oxide or titanium dioxide were mixed, and then stirred, thus manufacturing microtype scaffolds.

Moreover, microtype scaffolds prepared by mixing polyvinylalcohol and poly L-lactide was centrifuged and washed to freeze-dry, followed by being mixed with indium oxide or titanium dioxide to coat by heat treatment. A process of washing the coated microtype scaffolds by centrifugation and then freeze-drying them, was repeated to manufacture microtype scaffolds for cell culture, which have increased specific gravity.

Microtype scaffolds, which have increased specific gravity compared to existing microtype scaffolds for cell culture, can be manufactured by collecting microtype scaffolds which have higher specific gravity than that of Percoll using Percoll solution as specific gravity solution.

To improve cell stability during cell culture, and minimize risk factors in microtype scaffold manufacturing techniques and unpredictable variance in manufacturing microtype scaffolds for cell culture according to the present invention, it is preferable that the polymer material basically used is a biocompatible material and the inorganic compound used to increase specific gravity is biologically safe and at the same time the size thereof is less than 10 μm such that it has no significant effect on the polymer microtype scaffolds.

Since specific gravity of each ceramic is different (titanium dioxide: 4; barium titanate: 6.08; zircon: 2.1; zirconia dioxide: 5.56˜6.1; zinc oxide: 5.4˜5.7; silicon dioxide: 2.22.6; indium oxide: 7.19; tin oxide: 6.99.0), microtype scaffolds having desired specific gravity can be manufactured by varying the kinds of ceramics. Also, as specific gravity solutions have different specific gravity depending on the kinds thereof (Ficoll: 1.077; Percoll: 1.13; Histopaque: 1.077), microtype scaffolds having desired specific gravity can be manufactured according to specific gravity solution used.

In case where the diameter of said microtype scaffold for cell culture is more than 250 μm, cells are easily separated from the microtype scaffold upon cell separation but cell proliferation rate is low, and in case where the diameter of said microtype scaffold for cell culture is less than 10 μm, it is hard to separate cells from the microtype scaffold. Therefore, said microtype scaffold for cell culture preferably has a diameter of 10˜250 μm, more preferably 20˜150 μm, most preferably about 100 μm.

In another aspect, the present invention relates to a microtype scaffold for cell culture prepared by said method, which contains a biocompatible polymer and an inorganic compound for increasing specific gravity, and has a diameter of 10˜250 μm, as well as, a method for culturing cells, which comprises using said microtype scaffold for cell culture.

In the present invention, only adipocytes were illustrated as said cells, but other cells including fibroblasts, adult stem cells, preadipocytes, HeLa cells etc. can also be used without limitations as long as they are cells capable of adhering to microtype scaffolds to grow.

According to the present invention, when cells were cultured on said microtype scaffold for cell culture and then centrifuged, cells were easily separated from the microtype scaffold without a separate process.

### EXAMPLES

Hereinafter, the present invention will be described in more detail by specific examples. However, the present invention is not limited to these examples, and it is obvious to those of ordinary skill in the field of the present invention that numerous variations or modifications could be made within the spirit and scope of the present invention.

Especially, although the following examples illustrate only poly L-lactide (PLLA) as a biocompatible polymer, it is not limited thereto. Also, the following examples illustrate only indium oxide or titanium dioxide as an inorganic compound, but any material can be used without limitations as long as it is a material such as, ceramics, metals etc., which can increase specific gravity of microtype scaffolds.

The following examples illustrate only adipocytes as cells which can be used, but any type of cells, including fibroblasts, adult stem cells, preadipocytes, HeLa cells etc., can be used without limitations as long as they are cells that can adhere to microtype scaffolds to grow.

Moreover, the following examples illustrate only titanium dioxide as an inorganic compound responding to light, but any compound can be used without limitations as long as it is a compound, such as zinc oxide, tin oxide etc., which responds to light.

### Example 1

### Manufacture of Microtype Scaffolds for Cell Culture

### 1-1: Manufacture of Microtype Scaffolds for Cell Culture

To manufacture microtype scaffolds for cell culture having a high specific gravity, after poly L-lactide (PLLA) was dissolved in dichloromethane and mixed with indium oxide (In₂O₃) or titanium dioxide (TiO₂) to stir at 6,000 rpm for 3 min, 1% polyvinylalcohol (PVA) dissolved in distilled water was added thereto and stirred for 24 hrs, thus manufacturing microtype scaffolds. The microtype scaffolds were washed five times with PBS, followed by centrifugation at 3,000 rpm for 5 min, and then freeze-dried for 2 days.

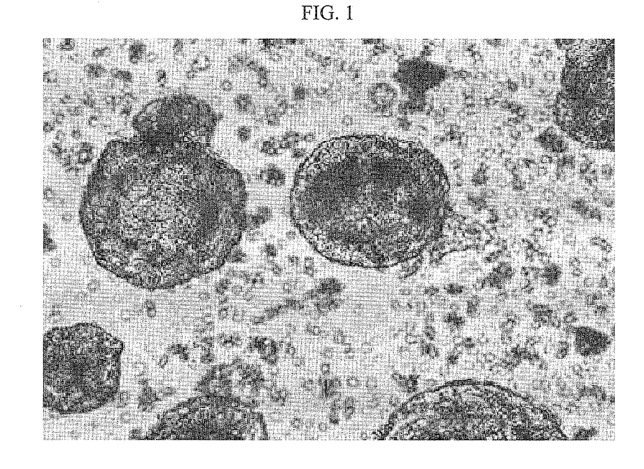

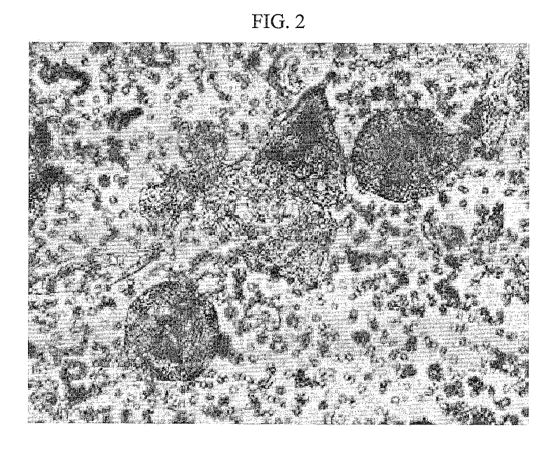

Microtype scaffolds for cell culture having an increased specific gravity were obtained by collecting microtype scaffolds having specific gravity of more than 1˜1.3 which is a specific gravity of Percoll through discontinuous density gradient method using Percoll. As a result of observing the obtained microtype scaffolds for cell culture by SEM, it was confirmed that particle size was 20˜150 μm (FIG. 1 and FIG. 2).

### 1-2: Manufacture of Microtype Scaffolds for Cell Culture

To manufacture microtype scaffolds for cell culture having a high specific gravity, 1% polyvinylalcohol (PVA) dissolved in distilled water, poly L-lactide (PLLA) dissolved in dichloromethane and indium oxide (In₂O₃) or titanium dioxide (TiO₂) were mixed with each other and stirred for 24 hrs, thus manufacturing microtype scaffolds. The obtained microtype scaffolds were washed 5 times with PBS, followed by centrifugation at 3,000 rpm for 5 min, and then freeze-dried for 2 days.

Microtype scaffolds for cell culture having an increased specific gravity were obtained by collecting microtype scaffolds having specific gravity of more than 1˜1.3 which is a specific gravity of Percoll through discontinuous density gradient method using Percoll. As a result of observing the obtained microtype scaffolds for cell culture by SEM, it was confirmed that particle size was 20˜150 μm.

### 1-3: Manufacture of Microtype Scaffolds for Cell Culture

Microtype scaffolds were manufactured by mixing 1% polyvinylalcohol (PVA) dissolved in distilled water and poly L-lactide (PLLA) dissolved in dichloromethane for 24 hrs. After the microtype scaffolds were washed 5 times with PBS, followed by centrifugation at 3,000 rpm for 5 min and freeze-dried for 2 days, they were mixed with indium oxide (In₂O₃) or titanium dioxide (TiO₂), and then they were coated by heat treatment. The process of washing microtype scaffolds 5 times with PBS, followed by centrifugation at 3,000 rpm for 5 min, and then freeze-drying for 2 days, was repeated.

Microtype scaffolds for cell culture having an increased specific gravity were obtained by collecting microtype scaffolds having specific gravity of more than 1˜1.3 which is a specific gravity of Percoll through discontinuous density gradient method using Percoll. As a result of observing the obtained microtype scaffolds for cell culture by SEM, it was confirmed that particle size was 20˜150 μm.

### Example 2

### Isolation Efficiency of Cells

Adipose tissue was separated from female breast tissue obtained from Breast Cancer Center, Seoul National University and washed with PBS. After the tissue was cut finely and digested by adding collagenase type 1 (1 mg/ml), it was centrifuged. Supernatant was sucked off and adipocytes were obtained from pellets left in the bottom.

The obtained cells were introduced into DMEM medium (4.00 mM L-glutamine, 4500 mg/L glucose, sodium pyruvate, distilled water) containing 10% fetal bovine serum (FBS) and 1% lipopolysaccharide (LPS) and the above prepared microtype scaffolds for cell culture, and shaken 3˜4 times to attach the cells to the microtype scaffolds for cell culture, and then cultured. A process of additionally introducing the above prepared microtype scaffolds for cell culture and the medium when primary cell culture is completed, was repeated.

Cells adhered to said microtype scaffolds for cell culture were centrifuged to examine the extent of cell separation from the microtype scaffolds. As a result, it was confirmed that the cells were easily separated from the microtype scaffolds only by centrifugation.

### Example 3

### Regulation of Cell Adherence

After adipocytes were adhered to the microtype scaffolds for cell culture prepared using titanium dioxide and cultured by the method described in Example 2, the cells were irradiated with UV to separate from the microtype scaffolds. As a result, it was confirmed that the cells were easily separated compared to a control group which was not irradiated with UV. It indicates that cell adherence is regulated by radiation intensity.

### INDUSTRIAL APPLICABILITY

As described and proven above in detail, the present invention provides microtype scaffolds for cell culture having an increased specific gravity and a method for manufacturing thereof. In case where the inventive microtype scaffolds for cell culture is used, cell damage can be minimized by reducing cell separation time, and it is easy to recover cells due to a definite boundary layer.

Although a specific embodiment of the present invention has been described in detail, those skilled in the art will appreciate that this description is merely a preferred embodiment and is not construed to limit the scope of the present invention. Thus, the substantial scope of the present invention will be defined by the accompanying claims and equivalents thereof.

What is claimed is:

1. A method for manufacturing microtype scaffolds for cell culture, the method comprising the steps of:
(a) mixing an inorganic compound with a biocompatible polymer; and
(b) mixing a biocompatible polymer with the mixture obtained in the step (a), and then washing and drying, thus manufacturing microtype scaffolds.

2. The method for manufacturing microtype scaffolds for cell culture according to claim 1, which additionally comprises a step of: (c) collecting the microtype scaffolds by using a specific gravity solution.

3. A method for manufacturing microtype scaffolds for cell culture, the method comprising the steps of:
(a) mixing two or more biocompatible polymers; and
(b) mixing an inorganic compound with the mixture obtained in the step (a), and then washing and drying, thus manufacturing microtype scaffolds.

4. The method for manufacturing microtype scaffolds for cell culture according to claim 3, which additionally comprises a step of: (c) collecting the microtype scaffolds by using a specific gravity solution.

5. A method for manufacturing microtype scaffolds for cell culture, the method comprising the steps of:
(a) mixing two or more biocompatible polymers; and
(b) washing and drying the mixture obtained in the step (a), and then coating it with an inorganic compound, thus manufacturing microtype scaffolds.

6. The method for manufacturing microtype scaffolds for cell culture according to claim 5, which additionally comprises a step of: (c) collecting the microtype scaffolds by using a specific gravity solution.

7. The method for manufacturing microtype scaffolds for cell culture according to claims 1 to 6, wherein the biocompatible polymer is selected from the group consisting of poly lactic acid (PLA), poly L-lactic acid (PLLA), poly glycolic acid (PGA), poly lactic-co-glycolic acid (PLGA), polyvinylalcohol (PVA), collagen, alginate, chitosan, fluorine resin (teflon), agar gel and polyacrylamide.

8. The method for manufacturing microtype scaffolds for cell culture according to claims 1 to 6, wherein the inorganic compound is selected from the group consisting of ceramic or metal.

9. The method for manufacturing microtype scaffolds for cell culture according to claim 8, wherein the ceramic is selected from the group consisting of hydroxyapatite (Ca₁₀(PO₄)₆(OH)₃), titanium dioxide (TiO₂), barium titanate (BiTiO₃), zircon (ZrSiO₄), zirconia dioxide (ZrO₂), iron oxide, zinc oxide (ZnO), silicon dioxide (SiO₂), indium oxide (In₂O₃) and tin oxide (SnO₂).

10. The method for manufacturing microtype scaffolds for cell culture according to claim 8, wherein the metal is selected from the group consisting of calcium, phosphorus, titanium, zirconium (Zr), iron (Fe), zinc, silicon, indium (In) and tin (Ti).

11. The method for manufacturing microtype scaffolds for cell culture according to claims 1 to 6, wherein the microtype scaffolds for cell culture are in the form of microbeads having a diameter of 10˜250 μm.

12. The method for manufacturing microtype scaffolds for cell culture according to claim 11, wherein the microtype scaffolds for cell culture are in the form of microbeads having a diameter of about 100 μm.

13. Microtype scaffolds for cell culture prepared by the method of any one claim among claims 1 to 6, which contain a biocompatible polymer and an inorganic compound for increasing specific gravity, and have a diameter of 10˜250 μm.

14. The microtype scaffolds for cell culture according to claim 13, wherein the biocompatible polymer is selected from the group consisting of poly lactic acid (PLA), poly L-lactic acid (PLLA), poly glycolic acid (PGA), poly lactic-co-glycolic acid (PLGA), polyvinylalcohol (PVA), collagen, alginate, chitosan, fluorine resin (teflon), agar gel and polyacrylamide.

15. The microtype scaffolds for cell culture according to claim 13, wherein the inorganic compound is selected from the group consisting of ceramic or metal.

16. The microtype scaffolds for cell culture according to claim 15, wherein the ceramic is selected from the group consisting of hydroxyapatite (Ca₁₀(PO₄)₆(OH)₃), titanium dioxide (TiO₂), barium titanate (BiTiO₃), zircon (ZrSiO₄), zirconia dioxide (ZrO₂), iron oxide, zinc oxide (ZnO), silicon dioxide (SiO₂), indium oxide (In₂O₃) and tin oxide (SnO₂).

17. The microtype scaffolds for cell culture according to claim 15, wherein the metal is selected from the group consisting of calcium, phosphorus, titanium, zirconium (Zr), iron (Fe), zinc, silicon, indium (In) and tin (Ti).

18. The microtype scaffolds for cell culture according to claim 13, wherein the inorganic compound is a compound responding to light.

19. The microtype scaffolds for cell culture according to claim 18, wherein the compound responding to light is selected from the group consisting of titanium dioxide (TiO₂), iron oxide, zinc oxide (ZnO) and tin oxide (SnO₂).

20. A method for culturing cells, the method comprises using microtype scaffolds for cell culture of claim 13.
